(19) United States
(12) Patent Application Publication
(10) Pub. No.: US 2025/0022585 A1
(43) Pub. Date: Jan. 16, 2025

(54) APPARATUS, SYSTEM AND METHOD TO FORWARD MEDICAL DATA FILES FROM A MEDICAL FACILITY NETWORK TO ANOTHER NETWORK

(71) Applicant: Exo Imaging, Inc., Santa Clara, CA (US)

(72) Inventors: Kyle Gavin Sawyer, Edmonton (CA); Zahin Hasan Prangon, Edmonton (CA); Ertugrul Alemdar, Somerville, MA (US)

(21) Appl. No.: 18/351,719

(22) Filed: Jul. 13, 2023

(51) Int. Cl. G16H 40/20; G16H 10/60; G16H 30/20; H04L 9/40

(52) U.S. Cl. CPC G16H 40/20; G16H 10/60; G16H 30/20; H04L 63/0428; H04L 63/166

(57) ABSTRACT

An apparatus, a system, a method, and a computer-readable storage medium. The apparatus including a memory storing logic corresponding to a medical data forwarding engine (MDFE), and one or more processors to execute the logic to perform operations including: accessing encoded medical information compliant with a medical signal handling protocol, the encoded medical information from a medical computing system of a medical facility network, wherein the encoded medical information includes one of a medical data file or a medical file fragment; preprocessing the encoded medical information by making the encoded medical information compliant with a networking communication protocol compatible with a web interface of a network external to the medical facility network (external network); and forwarding, after preprocessing, the encoded medical information to the external network based on the networking communication protocol.

### FIELD

Embodiments relate in general to the field of ultrasonic imaging probes.

### BACKGROUND

Ultrasound imaging is widely used in the fields of medicine and non-destructive testing and may have a diagnostic or a procedural purpose. An ultrasonic imaging probe generates ultrasound imaging data (UID) that may be shared through a communication network of a healthcare facility, such as a hospital. Such communication networks are typically provided with security features to minimize the chances of private patient information being accessed through malicious attacks.

### SUMMARY

According to some example embodiments, a medical data forwarding engine of a medical facility network is to receive encoded medical information, such as a medical data file or a medical file fragment (a fragment of the medical data file), to preprocess the encoded medical information to make it compatible with a secure networking communication protocol compatible with a web interface of an external network, and to forward the preprocessed encoded medical information to the cloud network based the a secure networking communication protocol.

### BRIEF DESCRIPTION OF THE DRAWINGS

The novel features of embodiments are set forth with particularity in the appended claims. A better understanding of the features and advantages of some embodiments will be obtained by reference to the following detailed description that sets forth illustrative embodiments, in which the principles of the invention are utilized, and the accompanying drawings (also “Figure” and “FIG.” herein), of which:

FIG. 1 is a block diagram of an ultrasound imaging probe in accordance with some embodiments.

FIG. 2 is a diagram of an ultrasound imaging environment in accordance with some embodiments.

FIG. 3 is a schematic diagram of an ultrasound imaging probe in accordance with some embodiments.

FIG. 4 depicts a medical file exchange environment according to some embodiments.

FIG. 5 depicts a flowchart of a process according to an embodiment.

### DETAILED DESCRIPTION

Because medical data file transfer and storage protocols such as DICOM are not secure enough for communication outside of a medical facility's network (e.g., they typically do not involve encryption), a medical facility network that stores or generates medical data files does not have a public IP. Some embodiments provide mechanisms to securely and efficiently communicate information contained in medical data files between a medical facility network and an external network.

A “medical facility” as used herein may refer to any location where medical services are provided to a patient. A medical facility may refer to a relatively permanent medical facility, such as a hospital, a medical clinic, a medical practice, a radiology department, a cardiology department, etc. A medical facility may refer to a temporary medical facility, such as an outdoor or indoor location where medical services are provided to a patient on a makeshift basis.

A “medical facility network” as used herein may refer to a network of medical computing systems including one or more electronic end unit devices, such as smartphones, tablets, laptops, desktop computers, medical imaging devices such as ultrasound imaging systems, within one or more medical facilities. A medical facility network may include a network of part or of all medical computing systems at a medical facility. Medical data files may be communicated within a medical facility network.

A “medical computing system” as used herein may include any computing device that is to be used for a medical purpose, diagnostic or procedural.

A “medical imaging computing system” as used herein is a subset of a “medical computing system,” and may include any computing device that is to be used for medical imaging purposes and that is to generate medical imaging data, diagnostic or procedural. Examples of a medical imaging computing system include, an x-ray system, an ultrasound imaging system, a magnetic resonance imaging system, a computed tomography (CT) scanning system, a positron emission tomography (PET) scanning system, a single photon emission computed tomography (SPECT) scanning system, an optical coherence tomography (OCT) system, or an endoscopy system, to name a few.

A “medical data file” refers to a digital collection of medical data that is uniquely identifiable. For example, a medical data file may correspond to a still digital image, a video footage, or an audio recording. A medical data file may, for example, correspond to a Digital Imaging and Communications in Medicine Standard (DICOM) compliant file. DICOM is a protocol for the storage, transmission and retrieval of medical images and related information. DICOM may be used to exchange medical imaging data between different systems and devices within a secure private network, such as within a medical facility network. A medical data file may, for example, correspond to a Health Level Seven (HL7) compliant file. A medical data file may correspond to any medical information communication protocol, such as those for the communication of medical image data files.

A “medical file fragment” refers to a fragment or a portion of a medical data file.

“Encoded medical information” as used herein may refer to a “medical data file” or to a “medical file fragment.”

A “cloud network” as referred to herein may refer to a computer network that uses cloud computing technology to provide services over the internet. In a cloud network, resources such as servers, storage, and applications are made available to users or clients through the internet. A cloud network may be made up of a combination of public and private clouds, which are managed by a cloud service provider (CSP).

Although the instant description will provide details regarding ultrasound imaging systems (e.g., ultrasound imaging probes or ultrasound computing device), it is to be understood that embodiments are not so limited, and include within their scope the use of medical information from any medical computing system(s) of a medical facility network, for example from any medical image computing system(s) of a medical facility network.

Ultrasound imaging probes may be used to image internal tissue, bones, blood flow, or organs of human or animal bodies in a non-invasive manner. The images can then be displayed. To perform ultrasound imaging, the ultrasound imaging probes transmit an ultrasonic signal into the body and receive a reflected signal from the body part being imaged. Such ultrasound imaging probes include transducers and associated electronics, which may be referred to as transceivers or imagers, and which may be based on photo-acoustic or ultrasonic effects. Such transducers may be used for imaging and may be used in other applications as well. For example, the transducers may be used in medical imaging; flow measurements in arteries and pipes, can form speakers and microphone arrays; can perform lithotripsy; localized tissue heating for therapeutic; and highly intensive focused ultrasound (HIFU) surgery.

Additional aspects and advantages of some embodiments will become readily apparent to those skilled in this art from the instant detailed description, wherein only illustrative embodiments are shown and described. As will be realized, some embodiments are capable of achieving other, different goals, and their several details are capable of modifications in various obvious respects, all without departing from the disclosure. Accordingly, the drawings and description are to be regarded as illustrative in nature, and not as restrictive.

Traditionally, imaging probes such as ultrasound imagers used in medical imaging use piezoelectric (PZT) materials or other piezo ceramic and polymer composites. Such imaging probes may include a housing to house the transducers with the PZT material, as well as other electronics that form and display the image on a display unit. To fabricate the bulk PZT elements or the transducers, a thick piezoelectric material slab may be cut into large rectangular shaped PZT elements. These rectangular-shaped PZT elements may be expensive to build, since the manufacturing process involves precisely cutting generally the rectangular-shaped thick PZT or ceramic material and mounting it on substrates with precise spacing. Further, the impedance of the transducers is much higher than the impedance of the body tissue, which can affect performance.

Still further, such thick bulk PZT elements can require very high voltage pulses, for example 100 volts (V) or more to generate transmission signals. This high drive voltage can sometimes result in high power dissipation since the power dissipation in the transducers is proportional to the square of the drive voltage. This high power dissipation generates heat within the ultrasound imaging probe such that cooling arrangements are necessitated. These cooling systems increase the manufacturing costs and weights of the ultrasound imaging probes which makes the ultrasound imaging probes more burdensome to operate.

Some embodiments may be utilized in the context of imaging probes that utilize either piezoelectric micromachined ultrasound transducer (pMUT) or capacitive micromachined ultrasonic transducer (cMUT) technologies, as described in further detail herein.

In general, MUTs, such as both cMUTs and pMUTs, include a diaphragm (a thin membrane attached at its edges, or at some point in the interior of the probe), whereas a “traditional,” bulk PZT element typically consists of a solid piece of material.

Piezoelectric micromachined ultrasound transducers (pMUTs) may be efficiently formed on a substrate leveraging various semiconductor wafer manufacturing operations. Semiconductor wafers may come in 6 inch, 8 inch, and 12 inch sizes and are capable of housing hundreds of transducer arrays. These semiconductor wafers start as a silicon substrate on which various processing operations are performed. An example of such an operation is the formation of SiO2 layers, also known as insulating oxides. Various other operations such as the addition of metal layers to serve as interconnects and bond pads are performed to allow connection to other electronics. Yet another example of a machine operation is the etching of cavities. Compared to the conventional transducers having bulky piezoelectric material, pMUT elements built on semiconductor substrates are less bulky, are cheaper to manufacture, and have simpler and higher performance interconnection between electronics and transducers. As such, they provide greater flexibility in the operational frequency of the ultrasound imaging probe using the same, and potential to generate higher quality images. Frequency response may for example be expanded though flexibility of shaping the diaphragm and its active areas with piezo material.

In some embodiments, the ultrasound imaging probe includes an application specific integrated circuit (ASIC) that includes transmit drivers, sensor circuitry for received echo signals, and control circuitry to control various operations. The ASIC may be formed on the same or another semiconductor wafer. This ASIC may be placed in close proximity to pMUT or cMUT elements to reduce parasitic losses. As a specific example, the ASIC may be 50 micrometers (μm) or less away from the transducer array. In a broader example, there may be less than 100 μm separation between the 2 wafers or 2 die, where each wafer includes many die, and a die includes a transducer array in the transducer wafer and an ASIC array in the ASIC wafer. The array may have up to 10,000 or more individual elements. In some embodiments, the ASIC has matching dimensions relative to the pMUT or cMUT array and allows the devices to be stacked for wafer-to-wafer interconnection or transducer die on ASIC wafer or transducer die to ASIC die interconnection. Alternatively, the transducer can also be developed on top of the ASIC wafer using low temperature piezo material sputtering and other low temperature processing compatible with ASIC processing.

Wherever the ASIC and the transducer interconnect, according to one embodiment, the two may have similar footprints. More specifically, according to the latter embodiment, a footprint of the ASIC may be an integer multiple or divisor of the MUT footprint.

Regardless of whether the ultrasound imaging probe is based on pMUT or cMUT, an imaging probe according to some embodiments may include a number of transmit channels and a number of receive channels. Transmit channels are to drive the transducer elements with a voltage pulse at a frequencies the elements are responsive to. This may cause an ultrasonic waveform to be emitted from the elements, which waveform is to be directed towards a target (i.e. that which is to be imaged), such as toward an organ or other tissue in a body. In some examples, the ultrasound imaging probe with the array of transducer elements may make mechanical contact with the body using a gel in between the ultrasound imaging probe and the body. The ultrasonic waveform travels towards the target, i.e., an organ, and a portion of the waveform is reflected back to the transducer elements in the form of received/reflected ultrasonic energy where the received ultrasonic energy may be converted to an electrical energy within the ultrasound imaging probe. The received ultrasonic energy may be processed by a number of receive channels to convert the received ultrasonic energy to signals, and the signals may be processed by other circuitry to develop an image of the target for display based on the signals.

An embodiment of an ultrasound imaging probe includes a transducer array, and control circuitry including, for example, an application-specific integrated circuit (ASIC), and transmit and receive beamforming circuitry, and optionally additional control electronics.

In an embodiment, an imaging probe may include a handheld casing or handheld housing where transducers and associated electronic circuitries, such as a control circuitry and optionally a computing device are housed. The ultrasound imaging probe may also contain a battery to power the electronic circuitries.

Thus, some embodiments pertain to a portable imaging probe utilizing either pMUT elements or cMUT elements in a 2D array. In some embodiments, such an array of transducer elements is coupled to an application specific integrated circuit (ASIC) of the ultrasound imaging probe.

An ultrasound exam (or “scanning operation”) may be associated with one of a number of ultrasound presets. An individual ultrasound preset may be associated with one or more ultrasound settings. Each setting can be changed or controlled independently of other settings within a preset to improve ultrasonic imaging.

In the following description, for purposes of explanation, specific details are set forth in order to provide an understanding of the disclosure. It will be apparent, however, to one skilled in the art that the disclosure may be practiced without these details. Furthermore, one skilled in the art will recognize that examples of the present disclosure, described below, may be implemented in a variety of ways, such as a process, one or more processors (processing circuitry) of a control circuitry, one or more processors (or processing circuitry) of a computing device, a system, a device, or a method on a tangible computer-readable medium.

One skilled in the art shall recognize: (1) that certain fabrication operations may optionally be performed; (2) that operations may not be limited to the specific order set forth herein; and (3) that certain operations may be performed in different orders, including being done contemporaneously, and (4) operations may involve using Artificial Intelligence.

Elements/components shown in diagrams are illustrative of exemplary embodiments and are meant to avoid obscuring the disclosure. Reference in the specification to “one example,” “preferred example,” “an example,” “examples,” “an embodiment,” “some embodiments,” or “embodiments” means that a particular feature, structure, characteristic, or function described in connection with the example is included in at least one example of the disclosure and may be in more than one example. The appearances of the phrases “in one example,” “in an example,” “in examples,” “in an embodiment,” “in some embodiments,” or “in embodiments” in various places in the specification are not necessarily all referring to the same example or examples. The terms “include,” “including,” “comprise,” and “comprising” shall be understood to be open terms and any lists that follow are examples and not meant to be limited to the listed items. Any headings used herein are for organizational purposes only and shall not be used to limit the scope of the description or the claims. Furthermore, the use of certain terms in various places in the specification is for illustration and should not be construed as limiting.

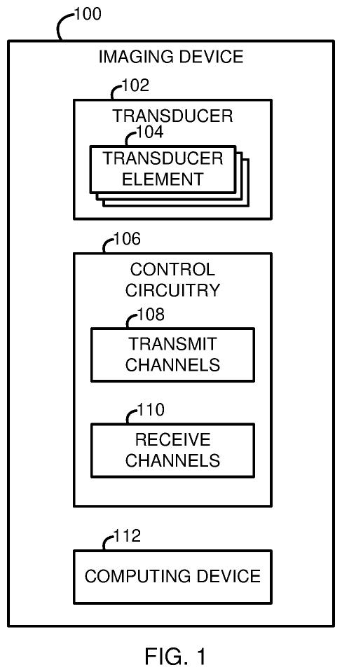

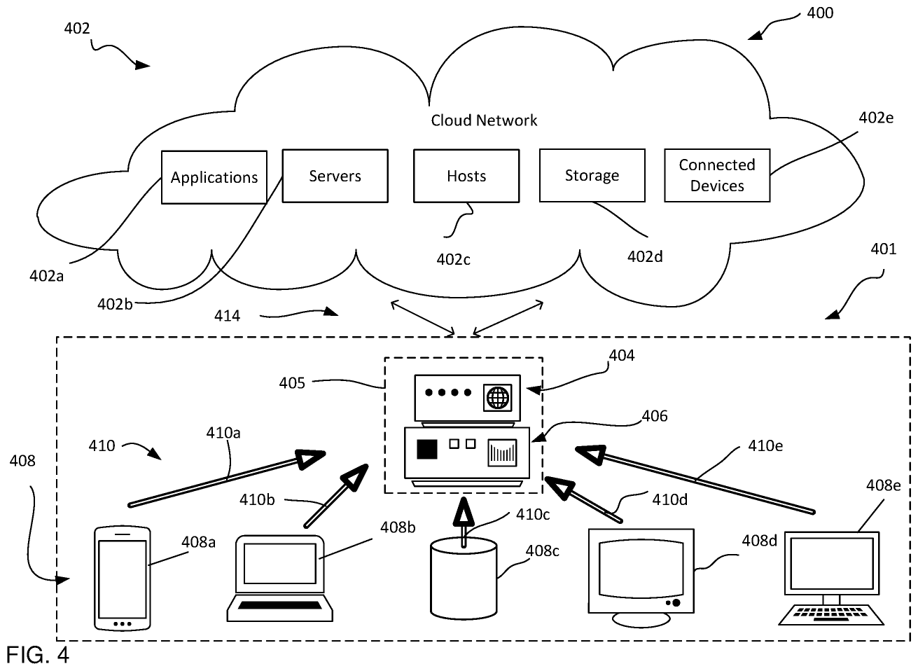

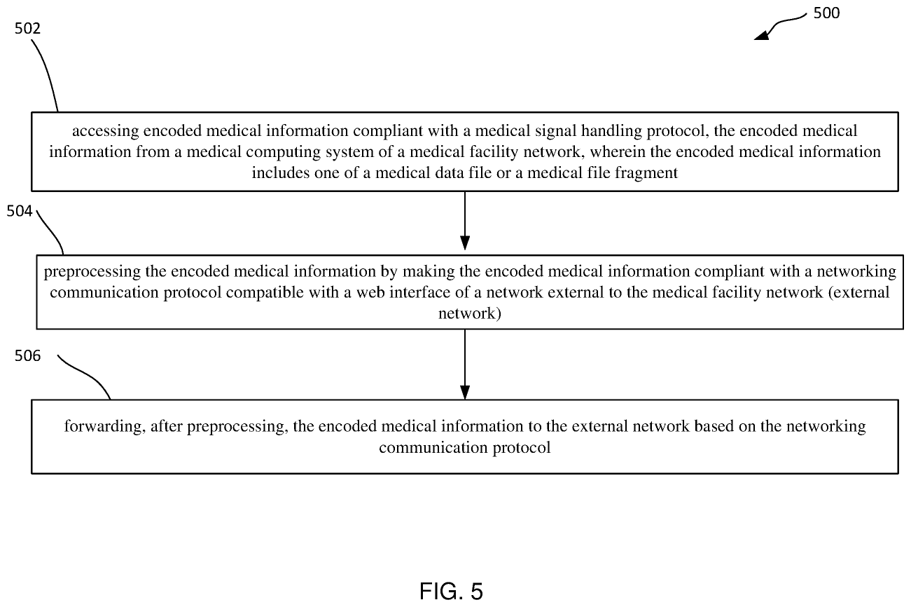

Reference will be made to FIGS. 1-3, which show devices and circuitries that may be used to implement some embodiments as described herein. Reference will further be made to FIGS. 4A and 4B which show an ultrasound imaging probe according to the state of the art. Reference will thereafter be made to FIGS. 5A and 5B, which show an ultrasound imaging probe according to one embodiment being held in two different manners.

Turning now to the figures, FIG. 1 is a block diagram of an ultrasound imaging probe 100 with a controller or control circuitry 106 controlling selectively alterable channels (108, 110) and having imaging computations performed on a computing device 112 according to principles described herein. As described above, the ultrasound imaging probe 100 may be used to generate an image of internal tissue, bones, blood flow, or organs of human or animal bodies. Accordingly, the ultrasound imaging probe 100 may transmit a signal into the body and receive a reflected signal from the body part being imaged. Such imaging probes may include either pMUT or cMUT, which may be referred to as transducers or imagers, which may be based on photo-acoustic or ultrasonic effects. The ultrasound imaging probe 100 may be used to image other targets as well. For example, the ultrasound imaging probe may be used in medical imaging; flow measurements in pipes, speaker, and microphone arrays; lithotripsy; localized tissue heating for therapeutic; and highly intensive focused ultrasound (HIFU) surgery.

In addition to use with human patients, the ultrasound imaging probe 100 may be used to acquire an image of internal organs of an animal as well. Moreover, in addition to imaging internal organs, the ultrasound imaging probe 100 may also be used to determine direction and velocity of blood flow in arteries and veins as in Doppler mode imaging and may also be used to measure tissue stiffness.

The ultrasound imaging probe 100 may be used to perform different types of imaging probe imaging. For example, the ultrasound imaging probe 100 may be used to perform one-dimensional imaging, also known as A-Scan, two-dimensional imaging, also known as B scan, three-dimensional imaging, also known as C scan, and Doppler imaging (that is, the use of Doppler ultrasound to determine movement, such as fluid flow within a vessel). The ultrasound imaging probe 100 may be switched to different imaging modes, including without limitation linear mode and sector mode, and electronically configured under program control.

To facilitate such imaging, the ultrasound imaging probe 100 includes one or more ultrasound transducers 102, each transducer 102 including an array of ultrasound transducer elements 104. Each ultrasound transducer element 104 may be embodied as any suitable transducer element, such as a pMUT or cMUT element. The transducer elements 104 operate to 1) generate the ultrasonic pressure waves that are to pass through the body or other mass and 2) receive reflected waves (received ultrasonic energy) off the target within the body, or other mass, to be imaged. In some examples, the ultrasound imaging probe 100 may be configured to simultaneously transmit and receive ultrasonic waveforms or ultrasonic pressure waves (pressure waves in short). For example, control circuitry 106 may be configured to control certain transducer elements 104 to send pressure waves toward the target being imaged while other transducer elements 104, at the same time, receive the pressure waves/ultrasonic energy reflected from the target, and generate electrical charges based on the same in response to the received waves/received ultrasonic energy/received energy.

In some examples, each transducer element 104 may be configured to transmit or receive signals at a certain frequency and bandwidth associated with a center frequency, as well as, optionally, at additional center frequencies and bandwidths. Such multi-frequency transducer elements 104 may be referred to as multi-modal elements 104 and can expand the bandwidth of the ultrasound imaging probe 100. The transducer element 104 may be able to emit or receive signals at any suitable center frequency, such as about 0.1 to about 100 megahertz.

To generate the pressure waves, the ultrasound imaging probe 100 may include a number of transmit (Tx) channels 108 and a number of receive (Rx) channels 110. The transmit channels 108 may include a number of components that drive the transducer 102, i.e., the array of transducer elements 104, with a voltage pulse at a frequency that they are responsive to. This may cause an ultrasonic waveform to be emitted from the transducer elements 104 towards a target to be imaged.

According to some embodiments, an ultrasonic waveform may include one or more ultrasonic pressure waves transmitted from one or more corresponding transducer elements of the ultrasound imaging probe substantially simultaneously.

The ultrasonic waveform travels towards the target to be imaged (target) and a portion of the waveform is reflected back to the transducer 102, which converts it to an electrical energy through a piezoelectric effect. The receive channels 110 collect electrical energy thus obtained, and process it, and send it for example to the computing device 112, which develops or generates an image that may be displayed.

In some examples, the number of transmit channels 108 and receive channels 110 in the ultrasound imaging probe 100 may remain constant, although the coupling of respective transducer elements to the transmit channels 108 and receive channels 110 may vary, for example based on coupling schemes dictated by the control circuitry. A coupling of the transmit and receive channels to the transducer elements may be, in one embodiment, controlled by control circuitry 106. In some examples, for example as shown in FIG. 1, the control circuitry may include the transmit channels 108 and the receive channels 110. For example, the transducer elements 104 of a transducer 102 may be formed into a two-dimensional spatial array with N columns and M rows. In a specific example, the two-dimensional array of transducer elements 104 may have 128 columns and 32 rows. In this example, the ultrasound imaging probe 100 may have up to 128 transmit channels 108 and up to 128 receive channels 110. In this example, each transmit channel 108 and receive channel 110 may be coupled to multiple or single pixels 104. For example, depending on the imaging mode (for example, whether a linear mode where a number of transducers transmit ultrasound waves in a same spatial direction, or a sector mode, where a number of transducers transmit ultrasound waves in different spatial directions), each column of transducer elements 104 may be coupled to a single transmit channel 108 and a single receive channel (110). In this example, the transmit channel 108 and receive channel 110 may receive composite signals, which composite signals combine signals received at each transducer element 104 within the respective column. In another example, i.e., during a different imaging mode, each transducer element 104 may be coupled to its dedicated transmit channel 108 and its dedicated receive channel 110. In some embodiments, a transducer element 104 may be coupled to both a transmit channel 108 and a receive channel 110. For example, a transducer element 104 may be adapted to create and transmit an ultrasound pulse and then detect the echo of that pulse in the form of converting the reflected ultrasonic energy into electrical energy.

The control circuitry 106 may be embodied as any circuit or circuits configured to perform the functions described herein. For example, the control circuitry 106 may be embodied as or otherwise include an application specific integrated circuit (ASIC), a field programmable gate array (FPGA), a system-on-a-chip, a processor and memory, a voltage source, a current source, one or more amplifiers, one or more digital-to-analog converters, one or more analog-to-digital converters, etc.

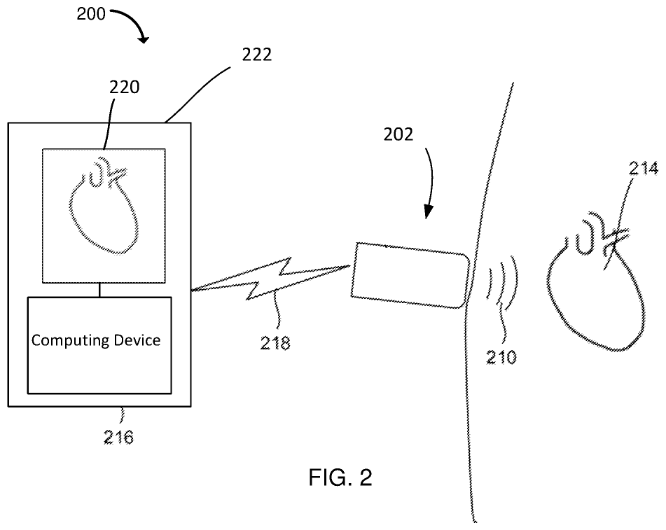

The illustrative computing device 112 may be embodied as any suitable computing device including any suitable components, such as one or more processors (i.e. one or more processing circuitries), one or more memory circuitries, one or more communication circuitries, one or more batteries, one or more displays, etc. In one embodiment, the computing device 112 may be integrated with the control circuitry 106, transducers 102, etc., into a single microelectronic package or single chip, or a single system on a chip (SoC), or a single ultrasound imaging probe housing as suggested for example in the embodiment of FIG. 1. In other embodiments, some or all of the computing devices may be in a separate microelectronic package from the control circuitry, or in a separate device distinct from the ultrasound imaging probe such as an ultrasound imaging probe, as suggested for example in the embodiment of in FIG. 2 as will be described in further detail below.

Each transducer element may have any suitable shape such as, square, rectangle, ellipse, or circle. The transducer elements may be arranged in a two-dimensional array arranged in orthogonal directions, such as in N columns and M rows as noted herein or may be arranged in an asymmetric (or staggered) rectilinear array.

Transducer elements 104 may have associated transmit driver circuits of associated transmit channels, and low noise amplifiers of associated receive channels. Thus, a transmit channel may include transmit drivers, and a receive channel may include one or more low noise amplifiers. For example, although not explicitly shown, the transmit and receive channels may each include multiplexing and address control circuitry to enable specific transducer elements and sets of transducer elements to be activated, deactivated or put in low power mode. It is understood that transducers may be arranged in patterns other than orthogonal rows and columns, such as in a circular fashion, or in other patterns based on the ranges of ultrasonic waveforms to be generated therefrom.

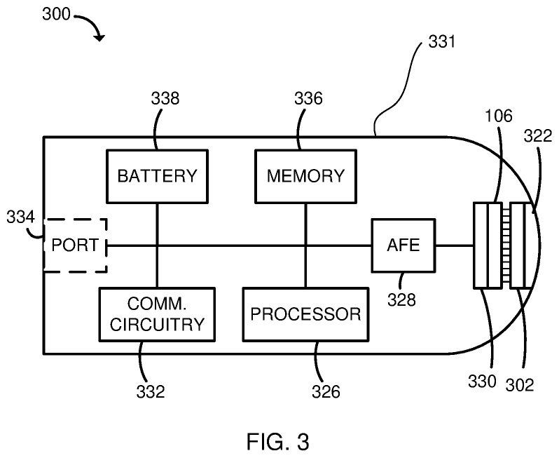

FIG. 2 is a diagram of an imaging environment including an imaging environment 200 with selectively configurable characteristics, according to an embodiment. The imaging environment of FIG. 2 may include an ultrasound imaging probe 202 (which may be similar to ultrasonic imaging probe 300 described below in the context of FIG. 3) and a computing system 222 which includes a computing device 216 and a display 220 coupled to the computing device, as will be described in further detail below.

As depicted in FIG. 2, the computing device 216 may, according to one embodiment, and unlike the embodiment of FIG. 1, be physically separate from the ultrasound imaging probe 220. For example, the computing device 216 and display device 220 may be disposed within a separate device (in this context, the shown computing system 222, physically separate from imaging probe 202 during operation) as compared with the components of the ultrasound imaging probe 202. The computing system 222 may include a mobile device, such as cell phone or tablet, or a stationary computing device, which can display images to a user. In another example, as shown in FIG. 1 for example, the display device, the computing device, and associated display, may be part of the ultrasound imaging probe 202 (now shown). That is, the ultrasound imaging probe 100, computing device 216, and display device 220 may be disposed within a single housing.

In one example, the computing system, such as computing system 222 of FIG. 2, may include a host processor device coupled to the computing device; a display communicatively coupled to a host processor; a network interface communicatively coupled to the host processor; or a battery to power the system.

A “computing device” as referred to herein may, in some embodiments, be configured to generate signals to at least one of cause an image of the target to be displayed on a display, or cause information regarding the image to be communicated to a user.

As depicted, the ultrasound imaging probe 202 is configured to generate and transmit, via the transmit channels (FIG. 1, 108), pressure waves 210 toward a target, such as a heart 214, in a transmit mode/process. The internal organ, or other target to be imaged, may reflect a portion of the pressure waves 210 toward the ultrasound imaging probe 202 which may receive, via a transducer (such as transducer 102 of FIG. 1), receive channels (FIG. 1, 110), control circuitry (FIG. 1, 106), the reflected pressure waves. The transducer may generate an electrical signal based on the received ultrasonic energy in a receive mode/process. A transmit mode or receive mode may be applicable in the context of imaging probes that may be configured to either transmit or receive, but at different times. However, as noted previously, some imaging probes according to embodiments may be adapted to be in both a transmit mode and a receive mode simultaneously. The system also includes a computing device 216 that is to communicate with the ultrasound imaging probe 100 through a communication channel, such as a wireless communication channel 218 as shown, although embodiments also encompass within their scope wired communication between a computing system and imaging probe. The ultrasound imaging probe 100 may communicate signals to the computing device 216 which may have one or more processors to process the received signals to complete formation of an image of the target. A display device 220 of the computing system 222 may then display images of the target using the signals from the computing device.

An imaging probe according to some embodiments may include a portable device, and/or a handheld device that is adapted to communicate signals through a communication channel, either wirelessly (using a wireless communication protocol, such as an IEEE 802.11 or Wi-Fi protocol, a Bluetooth protocol, including Bluetooth Low Energy, a mmWave communication protocol, or any other wireless communication protocol as would be within the knowledge of a skilled person) or via a wired connection such as a cable (such as USB2, USB 3, USB 3.1, and USB-C) or such as interconnects on a microelectronic device, with the computing device. In the case of a tethered or wired connection, the ultrasound imaging probe may include a port for receiving a cable connection of a cable that is to communicate with the computing device. In the case of a wireless connection, the ultrasound imaging probe 100 may include a wireless transceiver to communicate with the computing device 216.

It should be appreciated that, in various embodiments, different aspects of the disclosure may be performed in different components. For example, in one embodiment, the ultrasound imaging probe may include circuitry (such as the channels) to cause ultrasound waveforms to be sent and received through its transducers, while the computing device may be adapted to control such circuitry to the generate ultrasound waveforms at the transducer elements of the ultrasound imaging probe using voltage signals, and further a processing of the received ultrasonic energy.

FIG. 3 represents a view of an imaging probe according to some embodiments, as will be described in further detail below.

As seen in FIG. 3, the ultrasonic imaging probe 300 may include a handheld casing or housing 331 where transducers 302 and associated electronics are housed. The ultrasound imaging probe may also contain a battery 338 to power the electronics. FIG. 3 thus shows an embodiment of a portable imaging probe capable of 2D and 3D imaging using pMUTs in a 2D array, optionally built on a silicon wafer. Such an array coupled to an application specific integrated circuit (ASIC) 106 with electronic configuration of certain parameters, enables a higher quality of image processing at a lower cost than has been previously possible. Further by controlling certain parameters, for example the number of channels used, power consumption may be altered, and temperature may be changed.

FIG. 3 is a schematic diagram of an imaging probe such as an ultrasonic imaging probe 300 with selectively adjustable features, according to some embodiments. The ultrasonic imaging probe 300 may be similar to imaging probe 100 of FIG. 1, or to imaging probe 202 of FIG. 2, by way of example only. FIG. 3 depicts transducer(s) 302 of the ultrasonic imaging probe 300. As described above, the transducer(s) 302 may include arrays of transducer elements (FIG. 1, 104) that are adapted to transmit and receive pressure waves (FIG. 2, 210). In some examples, the ultrasonic imaging probe 300 may include a coating layer 322 that serves as an impedance matching interface between the transducers 302 and the human body, or other mass or tissue through which the pressure waves (FIG. 2, 210) are transmitted. In some cases, the coating layer 322 may serve as a lens when designed with the curvature consistent with focal length desired.

The ultrasonic imaging probe 300 housing 331 may be embodied in any suitable form factor. In some embodiments, part of the ultrasonic imaging probe 300 that includes the transducers 302 may extend outward from the rest of the ultrasonic imaging probe 300. The ultrasonic imaging probe 300 may be embodied as any suitable ultrasonic medical probe, such as a convex array probe, a micro-convex array probe, a linear array probe, an endovaginal probe, endorectal probe, a surgical probe, an intraoperative probe, etc.

In some embodiments, the user may apply gel on the skin of a living body before a direct contact with the coating layer 322 so that the impedance matching at the interface between the coating layer 322 and the human body may be improved. Impedance matching reduces the loss of the pressure waves (FIG. 2, 210) at the interface and the loss of the reflected wave travelling toward the ultrasonic imaging probe 300 at the interface.

In some examples, the coating layer 322 may be a flat layer to maximize transmission of acoustic signals from the transducer(s) 102 to the body and vice versa. The thickness of the coating layer 322 may be a quarter wavelength of the pressure wave (FIG. 2, 210) to be generated at the transducer(s) 102.

The ultrasonic imaging probe 300 also includes a control circuitry 106, such as one or more processors, optionally in the form of an application-specific integrated circuit (ASIC chip or ASIC), for controlling the transducers 102. The control circuitry 106 may be coupled to the transducers 102, such as by way of bumps.

The ultrasonic imaging probe 300 includes sensor circuitry 335 coupled to the communication circuitry 332 and to the processor circuitry 326. The sensor circuitry 335 may include one or more sensor circuitries to sense one or more dynamic parameters of the ultrasonic imaging probe.

The ultrasonic imaging probe may also include one or more processors (or processing circuitries) 326 for controlling the components of the ultrasonic imaging probe 300. One or more processors 326 may be configured to, in addition to control circuitry 106, at least one of control an activation of transducer elements, process signals based on reflected ultrasonic waveforms from the transducer elements or generate signals to cause generation of an image of an target being imaged by one or more processors of a computing device, such as computing device 112 of FIG. 1 or 216 of FIG. 2. One or more processors 326 may further be adapted to perform other processing functions associated with the ultrasonic imaging probe.

The one or more processors 326 may be embodied as any type of processors 326. For example, the one or more processors 326 may be embodied as a single or multi-core processor(s), a single or multi-socket processor, a digital signal processor, a graphics processor, a neural network compute engine, an image processor, a microcontroller, a field programmable gate array (FPGA), or other processor or processing/controlling circuit.

The ultrasonic imaging probe 300 may also include circuitry 328, such as Analog Front End (AFE), for processing/conditioning signals. The analog front end 328 may be embodied as any circuit or circuits configured to interface with the control circuitry 106 and other components of the ultrasonic imaging probe, such as the processing circuitry 326. For example, the analog front end 328 may include, e.g., one or more digital-to-analog converters, one or more analog-to-digital converters, one or more amplifiers, etc.

The ultrasonic imaging probe may include a communication circuitry 332 for communicating data, including control signals, with an external device, such as the computing device (FIG. 2, 216), through for example an input/output (I/O) circuitry 334 of the probe 300. The communication circuitry 332 may, for example, include signal processing circuitry for signals communicated through the I/O circuitry. For example, the communication circuitry may include one or more of a baseband processor, a modem, digital signal processing (DSP) circuitry, an analog-to-digital converter (ADC), or an equalizer circuitry.

For example, the I/O circuitry 334 may include one or more ports for wired communication with the probe 300, or a wireless transceiver circuitry 335 for wireless communication with the probe 300. The wireless transceiver circuitry 335 may, for example, include one or more transmit (TX) circuitries to transmit signals from the ultrasonic imaging probe 300, and one or more receive (RX) circuitries to receive signals into the ultrasonic imaging probe 300. The TX and RX circuitries may include TX and RX ports within port 334, which may, for example, correspond to a USB port. The TX and RX circuitries may include TX chains and RX chains of one or more wireless transceivers. A TX chain or an RX chain may include, for example, one or more antennas, amplifiers, filters, and/or mixers.

The ultrasonic imaging probe 300 may include memory circuitry 336 for storing data. The memory circuitry 336 may be embodied as any type of volatile or non-volatile memory or data storage capable of performing the functions described herein. In operation, the memory circuitry 336 may store various data and software used during operation of the ultrasonic imaging probe 300 such as operating systems, applications, programs, libraries, and drivers.

In some examples, the ultrasonic imaging probe 300 may include a battery 338 for providing electrical power to the components of the ultrasonic imaging probe 300. The battery 338 may also include battery charging circuits which may be wireless or wired charging circuits (not shown). The ultrasonic imaging probe may include a gauge that indicates a battery charge consumed and is used to configure the ultrasonic imaging probe to optimize power management for improved battery life. Additionally or alternatively, in some embodiments, the ultrasonic imaging probe may be powered by an external power source, such as by plugging the ultrasonic imaging probe into a wall outlet.

Medical data files, such as those compliant with a medical signal handling protocol, such as Digital Imaging and Communications in Medicine (DICOM), may include any imaging data (including audio and video data), such as imaging data relating to an ultrasound imaging session. The ultrasound imaging session may for example be performed using any of the ultrasound imaging systems (e.g., ultrasound imaging probe or ultrasound computing system) as described in relation to FIGS. 1-3 above. A medical image data file may further include any other kind of medical image data file, such as an image file obtained from an X-ray session, a CT scan session, an MRI session, etc.

There are several different types of DICOM medical data files, each with a specific purpose or function within the medical imaging workflow. Some of the most common types of DICOM compliant medical image data files include, by way of example, a medical image data file may correspond to one or more of:
1. a single-frame image, such as an X-ray or a CT scan image;
2. a multi-frame image, which corresponds to a sequence of images, such as a time-series of ultrasound images or a spatial series for a 3D volume of a target scanned using CT or MRI imaging systems; multi-frame images can be stored and transferred as a single file or as a set of related files in a group;
3. a structured report, which is a text-based report generated by radiologists or other healthcare providers to document the results of a medical imaging exam; a structured report can include information such as the patient's medical history, exam findings, and recommendations for further evaluation or treatment; or
4. a presentation state, which contains information about how a medical image data should be displayed on a display, such as information about brightness, contrast, and/or zoom level, etc.

These are just a few examples of the different types of DICOM files that are used in medical imaging.

Some embodiments pertain to the sharing of medical data files from the secure environment of a medical facility network to an external (i.e., separate from the medical facility network) network, such as a cloud network. Some embodiments pertain to using data piping to share medical data files with the outside network.

As used herein, “data piping” of a data file by a device refers to transferring the data file on a fragment by fragment basis such that a first fragment input into the device is sent through the output of the device to another device or application before all fragments of the data file are received at the device.

Reference is now made to FIG. 4, which shows an environment 400 to exchange medical data files between a medical facility network 401 and another (external) network, such as a cloud network 402. The cloud network may include or implement, for example, applications 402a, servers 402b, hosts 402c, storage 402d and/or connected devices 402e.

The medical facility network 402 is shown as including a number of medical computing system components 408, such as a smartphone or tablet 408a, a laptop computer 408b, a computer storage device 408c, a computer monitor 408d or a desktop computer 408e. The medical facility network 402 may further include a routing system 405, which may include a web interface circuitry 404, and a medical data forwarding entity (MDFE) 406 as will be described in further detail below. The web interface circuitry 404 may include, by way of example, a network interface card (NIC), a modem for wired or wireless communication, such as a WiFi access point, or any other router for wired and/or wireless communication.

By way of example, the routing system 405 of medical facility network 402 may include a Picture Archiving and Communication System (PACS), a system that is used by medical facilities to store, manage, and transmit digital medical images and related patient information. Such medical images may have been generated from image acquisition devices, such as ultrasound, X-Ray, CT Scan or MRI imaging systems to name a few. A PACS system may include servers and workstations that are used to store, process, and display the images. The PACS system may also include software that enables healthcare professionals to access and manipulate the images, as well as tools for archiving and retrieving images, and for securely transmitting them to other healthcare providers as needed.

The medical computing system components represent components of a medical computing system as defined above. These medical computing system components 408 of the medical facility network 402 may communicate medical data files with the cloud network 402, for example by way of wired or wireless connections, through the PACS system 405. For example, the electronic end user devices 408 may, as shown in FIG. 4, may be able to communicate with the cloud network 402 through web interface device 404, such as a WiFi access point (AP), or through any other suitable web interface device (wired and/or wireless) than would allow communication with an external network.

Although the web interface circuitry 404 and the MDFE 406 are shown as being within a same component, such as a PACS system, embodiments include within their scope an implementation of web interface circuitry and of MDFE 406 functionality in any manner within a medical facility network, including in respective circuitries, in a same circuitry or device, in a distributed manner as implemented through multiple circuitries of the medical facility network, etc.

The medical computing system components 408 may communicate (e.g., send or receive) medical data files with the cloud network 402 by way of a medical data forwarding entity (MDFE) 406 according to some embodiments. MDFE 406 refers to functionality implemented in hardware to forward medical data files between the cloud network 402 and the medical facility network 401 in a seamless and secure manner.

The MDFE 406 may, according to one embodiment, perform operations including at least: receiving at an input thereof a medical file fragment that is compliant with a medical signal handling protocol (e.g., DICOM), and routing the file fragment to the cloud network 402 through an output thereof before receiving all file fragments of the medical data file at the input thereof. The MDFE 406 according to some embodiments therefore performs data piping with respect to a medical data file.

The MDFE 406 may communicate, route, forward, transfer, send, transmit, or otherwise convey therethrough, from an output thereof, encoded medical information, for example a file fragment or a medical data file, after receiving the file fragment or medical data file at an input thereof. The MDFE 406 may, according to an embodiment, further preprocess (e.g., change some information within) the encoded medical information prior to communicating, routing, forwarding, transferring, sending, transmitting, or otherwise conveying the encoded medical information therethrough. Still, let us suppose that a certain encoded medical information is received at the MDFE, preprocessed through the MDFE 406 such that some information therein is changed according to some embodiments, and then outputted at an output of the MDFE in any direction (to or from the cloud network). In such a case, the instant description will refer to “the” encoded medical information as being received at and thereafter communicated, routed, forwarded, transferred, sent, transmitted, or otherwise conveyed by the MDFE 406, even though some information (aspects) of “the” encoded may have changed between the input of the MDFE 406 and the output of the MDFE, as long as the medical payload (data representing the primary medical content (e.g., medical image information, medical video information, other diagnostic or medical procedural information)) conveys substantially the same information as between the input and the output of the MDFE.

It is noted that an “input” of the MDFE 406 and an “output” of the MDFE 406 may correspond to one of multiple input ports and/or one of multiple output ports of circuitry within which the MDFE 406 is implemented, and may further be used to refer to different ports of such circuitry depending on the direction of transfer of encoded medical information through the MDFE (to or from the cloud network).

As noted previously, the MDFE 406 may be implemented in a dedicated circuitry of the medical facility as suggested in FIG. 4, or it may be implemented in circuitry that is part of one or more components of the medical facility network, such as circuitry of web interface device 404, or circuitry of one or more medical computing system components 408. In the shown example, MDFE 406 is implemented in a MDFE 406 router circuitry which communicates with the web interface device 404.

Referring still to FIG. 4, any one of communications 410, that is, any one of communications 410a, 410b, 410c, 410d, may include either a medical file fragment, or the complete medical data file itself. It is possible for example, according to some embodiments, that one of the medical computing systems 408 may be sending an entire medical data file, and another of the medical computing systems 408 may be sending only a file fragment. Communications 410 in FIG. 4 are depicted schematically as double-sided arrows to suggest that MDFE 406 may either send or receive medical data files or fragments thereof to one or more of the medical computing systems 408, and vice versa.

According to one embodiment, the MDFE 406 may, for example, successively receive at its input, data files and/or medical file fragments from a medical computing system of the medical facility network 401, and route the successively received data files and file fragments from its output to the cloud network before receiving all file fragments of any given medical data file.

According to one embodiment, the MDFE 406 may, for example, send successively received medical data files to the cloud network while receiving other medical data files and/or fragments thereof.

According to one embodiment, the MDFE 406 may, for example, send the successively received file fragments to the cloud network while receiving other file fragments of the medical data file.

According to one embodiment, the MDFE 406 may receive encoded medical information at substantially a same speed as sending the encoded medical information, meaning that preprocessing of the encoded medical information within the MDFE 406, according to some embodiments, adds negligible latency to encoded medical information transfer through the MDFE.

The MDFE 406 may, according to one embodiment, perform operations including receiving encoded medical information, such as a medical data file or a medical file fragment, and preprocessing the encoded medical information prior to forwarding the encoded medical information to cloud network 402.

According to some embodiments, the MDFE 406 may preprocess encoded medical information as it is received within the MDFE 406 and without causing the encoded medical information to persist.

According to some embodiments, the MDFE 406 may preprocess a medical data file in its entirety before forwarding the same to the cloud network 402, or it may fragment the medical data file prior to preprocessing the same, as already described in more detail below. The MDFE 406 may further preprocess individual medical file fragments of a medical data file and forward said individual medical file fragments, optionally in a same order as the order in which the MDFE 406 received them, to the cloud network 406.

The MDFE 406 may decompress the encoded medical information as part of preprocessing the same, and re-compress the encoded medical information as part of preprocessing the same.

According to some embodiments, the MDFE 406 may preprocess the encoded medical information by at least one of using compression, decompression, appending routing/destination information, encryption, format conversion, making the encoded medical information compliant with a networking communication protocol, or anonymization.

For example, the MDFE 406 may preprocess the medical data file or the medical file fragment by making it compliant with communication with a web interface based on a secure networking communication protocol, and forwarding, at 414, the preprocessed encoded medical information to the cloud network 402 based on the secure networking communication protocol.

For example, the MDFE 406 may preprocess the encoded medical information by anonymizing same prior to forwarding, at 414, the encoded medical information to the cloud network 402.

For example, the MDFE 406 may fragment a medical data file into a plurality of medical file fragments prior to preprocessing the medical file fragments, and forwarding, at 414, the fragments to the cloud network 420.

Some of the above preprocessing mechanisms will be described in further detail below.

Preprocessing to Make Encoded Medical Information Compliant with a Secure Networking Communication Protocol for Communication with a Web Interface

According to an embodiment, preprocessing (e.g., processing prior to forwarding) encoded medical information (e.g., a medical data file or a file fragment thereof) may include changing the encoded medical information to make it compliant with communication with a web interface based on a secure networking communication protocol. After preprocessing, MDFE 406 may forward the preprocessed encoded medical information to the cloud network in the form of communications 414 based on the secure networking communication protocol. The MDFE 406 may, according to some embodiments, be adapted to preprocess encoded medical information to be compliant with any number of networking communication protocols based on parameters of a web interface at the network (e.g., cloud network) to receive the encoded medical information.

According to some embodiments, the secure networking communication protocol may include WebSocket or Hypertext Transport Protocol Secure (HTTPS).

WebSocket enables two-way communication between a client (e.g. a medical computing system 408) and a server (e.g., at cloud network 402) for example over a single, long-lived Transmission Control Protocol (TCP) connection. Unlike the traditional Hypertext Transport Protocol (HTTP) request/response model, WebSocket allows for real-time, low-latency, full-duplex communication between a client and a server, meaning that both the client and the server can send and receive data at any time without having to wait for a request or response. Aspects of WebSocket may overcome some of the limitations of HTTP, such as the need for frequent requests and responses, the inability to handle server-initiated data, and the need for multiple connections to handle real-time data streams. With WebSocket, a single connection may be used to send and receive data in real-time, making it well-suited for applications that require real-time data updates, such as chat applications, online gaming, and financial trading platforms. WebSocket may be used, according to some embodiments, to enable real-time communication between a server-side application (such as at the medical facility network 401) and a server (such as at cloud network 402).

HTTPS is a protocol for secure communication over the internet, used to protect the privacy and security of sensitive data such as information in medical data files. HTTPS may be essentially the same as the standard HTTP protocol used for transferring data between a web server and a web browser, but with an added layer of encryption to protect the data from unauthorized access or interception. HTTPS may use SSL (Secure Sockets Layer) or TLS (Transport Layer Security) encryption to encrypt the data being transmitted over the internet, making it much more difficult for unauthorized parties to intercept or read the data. According to an embodiment, when the web interface circuitry 404 connects to a website in order to access the cloud network 402 using HTTPS, a browser of the web interface circuitry and a server of the cloud network 402 may exchange a series of keys to establish a secure connection using an SSL/TLS handshake. Once the connection is established, all data transmitted between the browser and the server may be encrypted and decrypted at each end, ensuring that sensitive information is protected from unauthorized access.

Making encoded medical information compliant with a networking communication protocol may include, by way of example, at least one of encryption, compression, or format conversion, for at least some of the encoded medical information.

For example, encryption may, according to an embodiment, include encrypting at least some of the encoded medical information, for example using a TLS protocol, whether for WebSocket or HTTPS, or using an SSL/TLS protocol for HTTPS.

For example, compression may, according to an embodiment, include reducing a size of some or all of the encoded medical information using a compression algorithm. A larger medical data or file fragment may slow down communications at 414, for example depending on traffic conditions. To improve performance, the MDFE 406 may for example compress the digital image using algorithms compliant with the networking communication protocol being used, such as WebSocket or HTTPS. For example, for WebSocket or HTTPS, the MDFE 406 may compress the encoded medical information using JPEG compression or lossless compression.

For example, format conversion may include changing a file format of some or all of the encoded medical information, for example changing the file format from a first file format into a second (different) file format, where the second file format may include any one of the following image or video file formats by way of example: for image file formats: Joint Photographic Experts Group (JPEG), Portable Network Graphics (PNG), or Tagged Image File Format (TIFF), and for video file formats: MPEG, MPEG4 part 14 (MP4), Audio Video Interleave (AVI), QuickTime File Format (MOV), Windows Media Video (WMV) or Flash Video (FLV).

Preprocessing Including Anonymization of Encoded Medical Information Communication with a Web Interface

One embodiment may include anonymizing the medical data file, or one or more of the medical file fragments, prior to routing the same to the cloud network.

According to some embodiments, anonymizing may include deidentification, pseudonymization, data minimization, or implementation of differential privacy, to name some examples. De-identification may include removing any personally identifiable information (PII) from the encoded medical information, such a name, an address, a birthdate, a social security number, etc. De-identification may be implemented through techniques such as masking, hashing, and encryption. Pseudonymization may involve replacing identifiable information with a unique identifier or code. Data minimization may involve reducing the amount of personal information in the encoded medical information. Differential privacy may involve adding random noise to the data in the encoded medical information to protect individual privacy while still allowing for accurate analysis.

### Fragmentation

According to some embodiments, the MDFE 406 may determine a size or length of a received medical data file, for example by accessing the metadata thereof, and, based on a length of the medical data file, fragment the medical data file into a plurality of file fragments prior to routing the file fragments to the cloud network. For example, the MDFE 406 may compare a length of the medical data file with a threshold length, and if the length of the medical data file is greater than, or is greater than or equal to, the threshold length, the MDFE 406 may fragment the medical data file into file fragments. According to some embodiments, the threshold length may be configurable to the MDFE. According to some embodiments, the MDFE 406 may select from a list of threshold based on parameters of the medical data file, such as quality of service requirements, latency requirements, source, destination in the receiving (e.g., cloud) network, networking communication protocol to be used to send the data file to the receiving network associated with the medical data file.

For example, a DICOM medical data file may be divided, by MDFE 406, into one or more fragments, where each fragment may have a fixed size. Each fragment may include a header that identifies its position in the DICOM data file and other relevant information.

When the cloud network 402 at the receiving end receives the fragments, it may reassemble them back into an original DICOM file using the information provided in the fragment headers.

According to some embodiments, a medical file fragment, such as one compliant with DICOM, may include: a fragment header (e.g., information such as the fragment's length, offset, and index within the DICOM file), fragment data (e.g., actual data contained within the fragment, which may include image pixel data or other types of information such as annotations or waveforms), a fragment trailer (e.g., with additional information about the fragment, such as its checksum or other data integrity measures).

For example, to preprocess an encoded medical information, the MDFE 406 may access metadata from the encoded medical information, and determine one or more parts of the encoded medical information to be changed based on the metadata.

For example, the MDFE 406 may determine, from the metadata, data corresponding to one or more pixels of the encoded medical information (pixel data) to be changed, decode the pixel data, and reencode the pixel data for example by at least one of encryption, compression, anonymization (e.g., where the metadata indicates location of patient identifying information), or format conversion of the pixel data. For example, the MDFE 406 may determine, from the metadata, data corresponding to a format of the imaging data within the encoded medical information (e.g., JPEG, MPEG, or uncompressed), and perform format conversion of the imaging data into a different format prior to forwarding the encoded medical information to the cloud network. For example, the MDFE 406 may determine, from the metadata, a size or length of an encoded medical information, and, based on a length of the medical data file, fragment the medical data file into a plurality of file fragments prior to routing the file fragments to the cloud network.

According to some embodiments, the MDFE 406 may decode data corresponding to one or more pixels of the encoded medical information, determine, based on the decoded data, data of the encoded medical information to be changed, and preprocess such data prior to forwarding the encoded medical information to the cloud network 402. For example, the MDFE 406 may determine, from decoded data, data corresponding to pixels with patient identifying information, and anonymize pixel data where needed.

According to some embodiments, the MDFE 406 may determine the data of the encoded medical information to be changed based on preconfigured pixel data. For example, MDFE 406 may know that one or more preconfigured pixels correspond to patient identifying information. Based on such knowledge, the MDFE 406 may anonymize the preconfigured pixels.

According to some embodiments, a MDFE 406 may use information regarding a determination of parts of a first encoded medical information to be preprocessed in order to determine parts of a second encoded medical information to be preprocessed. For example, the MDFE 406 may determine, from a decoding of pixel data within a first ended medical information, which pixel blocks of the first encoded medical information contain patient medical information and need to be anonymized. The MDFE 406 may then determine to anonymize the same pixel blocks in one or more subsequent medical informations based on an assumption that the same pixel blocks of the one or more subsequent medical informations contain patient identifying information.

FIG. 5 shows a method 500 to be performed at an apparatus of a medical facility according to one embodiment. The method includes, at operation 502, accessing encoded medical information compliant with a medical signal handling protocol, the encoded medical information from a medical computing system of the medical facility network, wherein the encoded medical information includes one of a medical data file or a medical file fragment; at operation 504, preprocessing the encoded medical information by making the encoded medical information compliant with a networking communication protocol compatible with a web interface of a network external to the medical facility network (external network); and at operation 506, forwarding, after preprocessing, the encoded medical information to the external network based on the networking communication protocol.

The flow of FIG. 5 is merely representative of operations that may occur in particular embodiments. In other embodiments, additional operations may be performed by the components of the systems shown in FIGS. 1-4. Various embodiments of the present disclosure contemplate any suitable mechanisms for accomplishing the functions described herein. Some of the operations illustrated in FIG. 5 may be repeated, combined, modified, or deleted where appropriate. Additionally, operations may be performed in any suitable order without departing from the scope of particular embodiments.

A design may go through various stages, from creation to simulation to fabrication. Data representing a design may represent the design in a number of manners. First, as is useful in simulations, some hardware may be represented using a hardware description language (HDL) or another functional description language. Additionally, a circuit level model with logic and/or transistor gates may be produced at some stages of the design process. Furthermore, most designs, at some stage, reach a level of data representing the physical placement of various devices in the hardware model. In some implementations, such data may be stored in a database file format such as Graphic Data System II (GDS II), Open Artwork System Interchange Standard (OASIS), or similar format.

In any representation of the design, the data may be stored in any form of a machine readable medium. A memory or a magnetic or optical storage such as a disc may be the machine readable medium to store information transmitted via optical or electrical wave modulated or otherwise generated to transmit such information. When an electrical carrier wave indicating or carrying the code or design is transmitted, to the extent that copying, buffering, or re-transmission of the electrical signal is performed, a new copy is made. Thus, a communication provider or a network provider may store on a tangible, machine-readable medium, at least temporarily, an article, such as information encoded into a carrier wave, embodying techniques of embodiments of the present disclosure.

In various embodiments, a medium storing a representation of the design may be provided to a manufacturing system (e.g., a semiconductor manufacturing system capable of manufacturing an integrated circuit and/or related components). The design representation may instruct the system to manufacture a device capable of performing any combination of the functions described above. For example, the design representation may instruct the system regarding which components to manufacture, how the components should be coupled together, where the components should be placed on the device, and/or regarding other suitable specifications regarding the device to be manufactured.

“Circuitry” as used herein may refer to any combination of hardware with software, and/or firmware. As an example, a circuitry includes hardware, such as a micro-controller, associated with a non-transitory medium to store code adapted to be executed by the micro-controller. Therefore, reference to a circuitry, in one embodiment, refers to the hardware, which is specifically configured to recognize and/or execute the code to be held on a non-transitory medium. Furthermore, in another embodiment, use of a circuitry refers to the non-transitory medium including the code, which is specifically adapted to be executed by the microcontroller to perform predetermined operations. And as can be inferred, in yet another embodiment, the term circuitry (in this example) may refer to the combination of the microcontroller and the non-transitory medium. Often circuitry boundaries that are illustrated as separate commonly vary and potentially overlap. For example, a first and a second circuitry may share hardware, software, firmware, or a combination thereof, while potentially retaining some independent hardware, software, or firmware. In one embodiment, use of the term logic includes hardware, such as transistors, registers, or other hardware, such as programmable logic devices.

Logic may be used to implement any of the flows described or functionality of the various components described herein. “Logic” may refer to hardware, firmware, software and/or combinations of each to perform one or more functions. In various embodiments, logic may include a microprocessor or other processing element operable to execute software instructions, discrete logic such as an application-specific integrated circuit (ASIC), a programmed logic device such as a field programmable gate array (FPGA), a storage device containing instructions, combinations of logic devices (e.g., as would be found on a printed circuit board), or other suitable hardware and/or software. Logic may include one or more gates or other circuit components. In some embodiments, logic may also be fully embodied as software. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in storage devices.

Use of the phrase ‘to’ or ‘configured to,’ in one embodiment, refers to arranging, putting together, manufacturing, offering to sell, importing, and/or designing an apparatus, hardware, logic, or element to perform a designated or determined task. In this example, an apparatus or element thereof that is not operating is still ‘configured to’ perform a designated task if it is designed, coupled, and/or interconnected to perform said designated task. As a purely illustrative example, a logic gate may provide a 0 or a 1 during operation. But a logic gate ‘configured to’ provide an enable signal to a clock does not include every potential logic gate that may provide a 1 or 0. Instead, the logic gate is one coupled in some manner that during operation the 1 or 0 output is to enable the clock. Note once again that use of the term ‘configured to’ does not require operation, but instead focuses on the latent state of an apparatus, hardware, and/or element, wherein the latent state the apparatus, hardware, and/or element is designed to perform a particular task when the apparatus, hardware, and/or element is operating.

Furthermore, use of the phrases ‘capable of/to,’ and or ‘operable to,’ in one embodiment, refers to some apparatus, logic, hardware, and/or element designed in such a way to enable use of the apparatus, logic, hardware, and/or element in a specified manner. Note as above that use of to, capable to, or operable to, in one embodiment, refers to the latent state of an apparatus, logic, hardware, and/or element, where the apparatus, logic, hardware, and/or element is not operating but is designed in such a manner to enable use of an apparatus in a specified manner.

The embodiments of methods, hardware, software, firmware, or code set forth above may be implemented via instructions or code stored on a machine-accessible, machine readable, computer accessible, or computer readable medium which are executable by a processing element. A tangible non-transitory machine-accessible/readable medium includes any mechanism that provides (i.e., stores and/or transmits) information in a form readable by a machine, such as a computer or electronic system. For example, a non-transitory machine-accessible medium includes random-access memory (RAM), such as static RAM (SRAM) or dynamic RAM (DRAM); ROM; magnetic or optical storage medium; flash storage devices; electrical storage devices; optical storage devices; acoustical storage devices; other form of storage devices for holding information received from transitory (propagated) signals (e.g., carrier waves, infrared signals, digital signals); etc., which are to be distinguished from the non-transitory mediums that may receive information therefrom.

Instructions used to program logic to perform embodiments of the disclosure may be stored within a memory in the system, such as DRAM, cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, Compact Disc, Read-Only Memory (CD-ROMs), and magneto-optical disks, Read-Only Memory (ROMs), Random Access Memory (RAM), Erasable Programmable Read-Only Memory (EPROM), Electrically Erasable Programmable Read-Only Memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the computer-readable medium includes any type of tangible machine-readable medium suitable for storing or transmitting electronic instructions or information in a form readable by a machine (e.g., a computer).

Some embodiments include an apparatus including means to perform operations according to any of the method embodiments described herein.

Some example embodiments will now be described below.

### Examples

Illustrative examples of the technologies disclosed herein are provided below. An embodiment of the technologies may include any one or more, and any combination of, the examples described below.

Example 1 includes an apparatus including a memory storing logic corresponding to a medical data forwarding engine (MDFE), and one or more processors to execute the logic to perform operations including: accessing encoded medical information compliant with a medical signal handling protocol, the encoded medical information from a medical computing system of a medical facility network, wherein the encoded medical information includes one of a medical data file or a medical file fragment; preprocessing the encoded medical information by making the encoded medical information compliant with a networking communication protocol compatible with a web interface of a network external to the medical facility network (external network); and forwarding, after preprocessing, the encoded medical information to the external network based on the networking communication protocol.

Example 2 includes the subject matter of Example 1, wherein the operations include determining the networking communication protocol prior to preprocessing.

Example 3 includes the subject matter of Example 2, wherein the networking communication protocol includes at least one of WebSocket or Hypertext Transport Protocol Secure (HTTPS).

Example 4 includes the subject matter of any one of Examples 1-3, wherein the networking communication protocol allows full-duplex communication using a Transmission Control Protocol (TCP) connection.

Example 5 includes the subject matter of any one of Examples 1-3, wherein the networking communication protocol uses a Secure Socket Layer (SSL) encryption or a Transport Layer Security (TLS) encryption.

Example 6 includes the subject matter of any one of Examples 1-4, wherein the networking communication protocol uses a Secure Socket Layer (SSL) handshake or a Transport Layer Security (TLS) handshake.

Example 7 includes the subject matter of any one of Examples 1-5, wherein preprocessing further includes at least one of: compressing, decompressing, appending routing information, encrypting, decrypting, format conversion, or anonymizing.

Example 8 includes the subject matter of Example 6, wherein preprocessing that comprises format conversion includes changing a file format of the encoded medical information from a first file format to a second file format different from the first file format to make the encoded medical information compatible with the networking communication protocol.

Example 9 includes the subject matter of Example 8, wherein the first file format or the second file format includes one of: a Joint Photographic Experts Group (JPEG) format, a Portable Network Graphics (PNG) format, a Tagged Image File Format (TIFF) format, an MPEG format, a MPEG4 part 14 (MP4) format, an Audio Video Interleave (AVI) format, a QuickTime File Format (MOV) format, a Windows Media Video (WMV) format, or Flash Video (FLV) format.

Example 10 includes the subject matter of Example 7, wherein anonymizing includes at least one of: removing personal identifying information (PII), replacing PII with a unique identifier, reducing information in the PII, or adding noise to the encoded medical information.

Example 11 includes the subject matter of any one of Examples 1-10, wherein the operations further include determining, prior to preprocessing and based on at least one of metadata of the encoded medical information or pixel data of the encoded medical information, one or more parts of the encoded medical information to be preprocessed,

Example 12 includes the subject matter of any one of Examples 1-10, wherein the operations further include determining, prior to preprocessing and based on a preconfiguration of pixel data corresponding to one or more parts of the encoded medical information to be changed, the one or more parts of the encoded medical information.

Example 13 includes the subject matter of any one of Examples 11 or 12, wherein the one or more parts correspond to at least one of metadata of the encoded medical information or payload data of the encoded medical information.

Example 14 includes the subject matter of any one of Examples 11-13, wherein the encoded medical information is first encoded medical information, and the one or more parts are one or more first parts, the operations further including, after determining the one or more first parts, determining one or more second parts of a second encoded medical information based on a determination of the one or more first parts, preprocessing the one or more second parts to make the encoded medical information compliant with the networking communication protocol, and forwarding, after preprocessing the one or more second parts, the second encoded medical information to the external network based on the networking communication protocol.

Example 15 includes the subject matter of any one of Examples 1-14, wherein the operations further include receiving a medical data file from the medical facility network, and fragmenting the medical data file to obtain medical file fragments therefrom, the encoded medical information corresponding to one of the medical file fragments.

Example 16 includes the subject matter of Example 15, wherein fragmenting includes fragmenting based on a size of the medical data file.

Example 17 includes the subject matter of Example 15, wherein the one or more processors are one or more processors of a Picture Archiving and Communication System (PACS).

Example 18 includes the subject matter of any one of Examples 1-17, wherein the encoded medical information includes a first medical file fragment of a first medical data file, the operations further including routing the first medical file fragment and subsequent medical file fragments of the first medical data file on a fragment by fragment basis by routing the first medical file fragment to the external network before all fragments of the first medical data file are accessed by the one or more processor.

Example 19 includes the subject matter of Example 18, wherein the first medical data file is part of a plurality of medical data files from the medical facility network, and the first medical file fragment is part of a plurality of medical file fragments of at least some of the medical data files, the operations further including routing the medical file fragments of at least some of the plurality of medical data files, and remaining ones of the plurality of medical data files to the external network before receiving all file fragments of at least one of the plurality of medical data files.

Example 20 includes the subject matter of any one of Examples 1-19, wherein the medical signal handling protocol includes Digital Imaging and Communications in Medicine (DICOM).

Example 21 includes the subject matter of any one of Examples 1-20, wherein the medical data file is an imaging medical data file.

Example 22 includes a routing system including: a web interface circuitry; a memory storing logic corresponding a medical data forwarding engine (MDFE); and one or more processors coupled to the web interface circuitry to execute the logic to perform operations including: accessing encoded medical information compliant with a medical signal handling protocol, the encoded medical information from a medical computing system of a medical facility network, wherein the encoded medical information includes one of a medical data file or a medical file fragment; preprocessing the encoded medical information by making the encoded medical information compliant with a networking communication protocol compatible with a web interface of a network external to the medical facility network (external network); and forwarding, after preprocessing, and through the web interface circuitry, the encoded medical information to the external network based on the networking communication protocol.

Example 23 includes the subject matter of Example 22, wherein the operations include determining the networking communication protocol prior to preprocessing.

Example 24 includes the subject matter of Example 23, wherein the networking communication protocol includes at least one of WebSocket or Hypertext Transport Protocol Secure (HTTPS).

Example 25 includes the subject matter of any one of Examples 22-24, wherein the networking communication protocol allows full-duplex communication using a Transmission Control Protocol (TCP) connection.

Example 26 includes the subject matter of any one of Examples 22-24, wherein the networking communication protocol uses a Secure Socket Layer (SSL) encryption or a Transport Layer Security (TLS) encryption.

Example 27 includes the subject matter of any one of Examples 22-26, wherein the networking communication protocol uses a Secure Socket Layer (SSL) handshake or a Transport Layer Security (TLS) handshake.

Example 28 includes the subject matter of any one of Examples 22-27, wherein preprocessing further includes at least one of: compressing, decompressing, appending routing information, encrypting, decrypting, format conversion, or anonymizing.

Example 29 includes the subject matter of Example 28, wherein preprocessing that comprises format conversion includes changing a file format of the encoded medical information from a first file format to a second file format different from the first file format to make the encoded medical information compatible with the networking communication protocol.

Example 30 includes the subject matter of Example 29, wherein the first file format or the second file format includes one of: a Joint Photographic Experts Group (JPEG) format, a Portable Network Graphics (PNG) format, a Tagged Image File Format (TIFF) format, an MPEG format, a MPEG4 part 14 (MP4) format, an Audio Video Interleave (AVI) format, a QuickTime File Format (MOV) format, a Windows Media Video (WMV) format, or Flash Video (FLV) format.

Example 31 includes the subject matter of Example 28, wherein anonymizing includes at least one of: removing personal identifying information (PII), replacing PII with a unique identifier, reducing information in the PII, or adding noise to the encoded medical information.

Example 32 includes the subject matter of any one of Examples 22-31, wherein the operations further include determining, prior to preprocessing and based on at least one of metadata of the encoded medical information or pixel data of the encoded medical information, one or more parts of the encoded medical information to be preprocessed,

Example 33 includes the subject matter of any one of Examples 22-31, wherein the operations further include determining, prior to preprocessing and based on a preconfiguration of pixel data corresponding to one or more parts of the encoded medical information to be changed, the one or more parts of the encoded medical information.

Example 34 includes the subject matter of any one of Examples 32 or 33, wherein the one or more parts correspond to at least one of metadata of the encoded medical information or payload data of the encoded medical information.

Example 35 includes the subject matter of any one of Examples 32-34, wherein the encoded medical information is first encoded medical information, and the one or more parts are one or more first parts, the operations further including, after determining the one or more first parts, determining one or more second parts of a second encoded medical information based on a determination of the one or more first parts, preprocessing the one or more second parts to make the encoded medical information compliant with the networking communication protocol, and forwarding, after preprocessing the one or more second parts, the second encoded medical information to the external network based on the networking communication protocol.

Example 36 includes the subject matter of any one of Examples 22-35, wherein the operations further include receiving a medical data file from the medical facility network, and fragmenting the medical data file to obtain medical file fragments therefrom, the encoded medical information corresponding to one of the medical file fragments.

Example 37 includes the subject matter of Example 36, wherein fragmenting includes fragmenting based on a size of the medical data file.

Example 38 includes the subject matter of Example 36, wherein the one or more processors are one or more processing circuitries of a Picture Archiving and Communication System (PACS).

Example 39 includes the subject matter of any one of Examples 22-38, wherein the encoded medical information includes a first medical file fragment of a first medical data file, the operations further including routing the first medical file fragment and subsequent medical file fragments of the first medical data file on a fragment by fragment basis by routing the first medical file fragment to the external network before all fragments of the first medical data file are accessed by the one or more processor.

Example 40 includes the subject matter of Example 39, wherein the first medical data file is part of a plurality of medical data files from the medical facility network, and the first medical file fragment is part of a plurality of medical file fragments of at least some of the medical data files, the operations further including routing the medical file fragments of at least some of the plurality of medical data files, and remaining ones of the plurality of medical data files to the external network before receiving all file fragments of at least one of the plurality of medical data files.

Example 41 includes the subject matter of any one of Examples 22-40, wherein the medical signal handling protocol includes Digital Imaging and Communications in Medicine (DICOM).

Example 42 includes the subject matter of any one of Examples 22-41, wherein the medical data file is an imaging medical data file.

Example 43 includes a product including one or more tangible computer-readable non-transitory storage media comprising computer-executable instructions operable to, when executed by one or more processors, cause the one or more processors to implement operations comprising: accessing encoded medical information compliant with a medical signal handling protocol, the encoded medical information from a medical computing system of a medical facility network, wherein the encoded medical information includes one of a medical data file or a medical file fragment; preprocessing the encoded medical information by making the encoded medical information compliant with a networking communication protocol compatible with a web interface of a network external to the medical facility network (external network); and forwarding, after preprocessing, the encoded medical information to the external network based on the networking communication protocol.

Example 44 includes the subject matter of Example 43, wherein the operations include determining the networking communication protocol prior to preprocessing.

Example 45 includes the subject matter of Example 44, wherein the networking communication protocol includes at least one of WebSocket or Hypertext Transport Protocol Secure (HTTPS).

Example 46 includes the subject matter of any one of Examples 43-45, wherein the networking communication protocol allows full-duplex communication using a Transmission Control Protocol (TCP) connection.

Example 47 includes the subject matter of any one of Examples 43-45, wherein the networking communication protocol uses a Secure Socket Layer (SSL) encryption or a Transport Layer Security (TLS) encryption.

Example 48 includes the subject matter of any one of Examples 43-46, wherein the networking communication protocol uses a Secure Socket Layer (SSL) handshake or a Transport Layer Security (TLS) handshake.

Example 49 includes the subject matter of any one of Examples 43-47, wherein preprocessing further includes at least one of: compressing, decompressing, appending routing information, encrypting, decrypting, format conversion, or anonymizing.

Example 50 includes the subject matter of Example 48, wherein preprocessing that comprises format conversion includes changing a file format of the encoded medical information from a first file format to a second file format different from the first file format to make the encoded medical information compatible with the networking communication protocol.

Example 51 includes the subject matter of Example 50, wherein the first file format or the second file format includes one of: a Joint Photographic Experts Group (JPEG) format, a Portable Network Graphics (PNG) format, a Tagged Image File Format (TIFF) format, an MPEG format, a MPEG4 part 14 (MP4) format, an Audio Video Interleave (AVI) format, a QuickTime File Format (MOV) format, a Windows Media Video (WMV) format, or Flash Video (FLV) format.

Example 52 includes the subject matter of Example 49, wherein anonymizing includes at least one of: removing personal identifying information (PII), replacing PII with a unique identifier, reducing information in the PII, or adding noise to the encoded medical information.

Example 53 includes the subject matter of any one of Examples 43-52, wherein the operations further include determining, prior to preprocessing and based on at least one of metadata of the encoded medical information or pixel data of the encoded medical information, one or more parts of the encoded medical information to be preprocessed.

Example 54 includes the subject matter of any one of Examples 43-52, wherein the operations further include determining, prior to preprocessing and based on a preconfiguration of pixel data corresponding to one or more parts of the encoded medical information to be changed, the one or more parts of the encoded medical information.

Example 55 includes the subject matter of any one of Examples 53 or 54, wherein the one or more parts correspond to at least one of metadata of the encoded medical information or payload data of the encoded medical information.

Example 56 includes the subject matter of any one of Examples 53-55, wherein the encoded medical information is first encoded medical information, and the one or more parts are one or more first parts, the operations further including, after determining the one or more first parts, determining one or more second parts of a second encoded medical information based on a determination of the one or more first parts, preprocessing the one or more second parts to make the encoded medical information compliant with the networking communication protocol, and forwarding, after preprocessing the one or more second parts, the second encoded medical information to the external network based on the networking communication protocol.

Example 57 includes the subject matter of any one of Examples 43-56, wherein the operations further include receiving a medical data file from the medical facility network, and fragmenting the medical data file to obtain medical file fragments therefrom, the encoded medical information corresponding to one of the medical file fragments.

Example 58 includes the subject matter of Example 57, wherein fragmenting includes fragmenting based on a size of the medical data file.

Example 59 includes the subject matter of Example 57, wherein the one or more processors are one or more processing circuitries of a Picture Archiving and Communication System (PACS).

Example 60 includes the subject matter of any one of Examples 43-59, wherein the encoded medical information includes a first medical file fragment of a first medical data file, the operations further including routing the first medical file fragment and subsequent medical file fragments of the first medical data file on a fragment by fragment basis by routing the first medical file fragment to the external network before all fragments of the first medical data file are accessed by the one or more processor.

Example 61 includes the subject matter of Example 60, wherein the first medical data file is part of a plurality of medical data files from the medical facility network, and the first medical file fragment is part of a plurality of medical file fragments of at least some of the medical data files, the operations further including routing the medical file fragments of at least some of the plurality of medical data files, and remaining ones of the plurality of medical data files to the external network before receiving all file fragments of at least one of the plurality of medical data files.

Example 62 includes the subject matter of any one of Examples 43-61, wherein the medical signal handling protocol includes Digital Imaging and Communications in Medicine (DICOM).

Example 63 includes the subject matter of any one of Examples 43-62, wherein the medical data file is an imaging medical data file.

Example 64 includes a method to be implemented at a processing circuitry of a medical facility network, the method including: accessing encoded medical information compliant with a medical signal handling protocol, the encoded medical information from a medical computing system of the medical facility network, wherein the encoded medical information includes one of a medical data file or a medical file fragment; preprocessing the encoded medical information by making the encoded medical information compliant with a networking communication protocol compatible with a web interface of a network external to the medical facility network (external network); and forwarding, after preprocessing, the encoded medical information to the external network based on the networking communication protocol.

Example 65 includes the subject matter of Example 64, further including determining the networking communication protocol prior to preprocessing.

Example 66 includes the subject matter of Example 65, wherein the networking communication protocol includes at least one of WebSocket or Hypertext Transport Protocol Secure (HTTPS).

Example 67 includes the subject matter of any one of Examples 64-66, wherein the networking communication protocol allows full-duplex communication using a Transmission Control Protocol (TCP) connection.

Example 68 includes the subject matter of any one of Examples 64-66, wherein the networking communication protocol uses a Secure Socket Layer (SSL) encryption or a Transport Layer Security (TLS) encryption.

Example 69 includes the subject matter of any one of Examples 64-67, wherein the networking communication protocol uses a Secure Socket Layer (SSL) handshake or a Transport Layer Security (TLS) handshake.

Example 70 includes the subject matter of any one of Examples 64-68, wherein preprocessing further includes at least one of: compressing, decompressing, appending routing information, encrypting, decrypting, format conversion, or anonymizing.

Example 71 includes the subject matter of Example 69, wherein preprocessing that comprises format conversion includes changing a file format of the encoded medical information from a first file format to a second file format different from the first file format to make the encoded medical information compatible with the networking communication protocol.

Example 72 includes the subject matter of Example 71, wherein the first file format or the second file format includes one of: a Joint Photographic Experts Group (JPEG) format, a Portable Network Graphics (PNG) format, a Tagged Image File Format (TIFF) format, an MPEG format, a MPEG4 part 14 (MP4) format, an Audio Video Interleave (AVI) format, a QuickTime File Format (MOV) format, a Windows Media Video (WMV) format, or Flash Video (FLV) format.

Example 73 includes the subject matter of Example 70, wherein anonymizing includes at least one of: removing personal identifying information (PII), replacing PII with a unique identifier, reducing information in the PII, or adding noise to the encoded medical information.

Example 74 includes the subject matter of any one of Examples 64-73, further including determining, prior to preprocessing and based on at least one of metadata of the encoded medical information or pixel data of the encoded medical information, one or more parts of the encoded medical information to be preprocessed,

Example 75 includes the subject matter of any one of Examples 64-73, further including determining, prior to preprocessing and based on a preconfiguration of pixel data corresponding to one or more parts of the encoded medical information to be changed, the one or more parts of the encoded medical information.

Example 76 includes the subject matter of any one of Examples 74 or 75, wherein the one or more parts correspond to at least one of metadata of the encoded medical information or payload data of the encoded medical information.

Example 77 includes the subject matter of any one of Examples 74-76, wherein the encoded medical information is first encoded medical information, and the one or more parts are one or more first parts, the operations further including, after determining the one or more first parts, determining one or more second parts of a second encoded medical information based on a determination of the one or more first parts, preprocessing the one or more second parts to make the encoded medical information compliant with the networking communication protocol, and forwarding, after preprocessing the one or more second parts, the second encoded medical information to the external network based on the networking communication protocol.

Example 78 includes the subject matter of any one of Examples 64-77, further including receiving a medical data file from the medical facility network, and fragmenting the medical data file to obtain medical file fragments therefrom, the encoded medical information corresponding to one of the medical file fragments.

Example 79 includes the subject matter of Example 78, wherein fragmenting includes fragmenting based on a size of the medical data file.

Example 80 includes the subject matter of Example 78, wherein the one or more processors are one or more processing circuitries of a Picture Archiving and Communication System (PACS).

Example 81 includes the subject matter of any one of Examples 64-80, wherein the encoded medical information includes a first medical file fragment of a first medical data file, the operations further including routing the first medical file fragment and subsequent medical file fragments of the first medical data file on a fragment by fragment basis by routing the first medical file fragment to the external network before all fragments of the first medical data file are accessed by the one or more processor.

Example 82 includes the subject matter of Example 81, wherein the first medical data file is part of a plurality of medical data files from the medical facility network, and the first medical file fragment is part of a plurality of medical file fragments of at least some of the medical data files, the operations further including routing the medical file fragments of at least some of the plurality of medical data files, and remaining ones of the plurality of medical data files to the external network before receiving all file fragments of at least one of the plurality of medical data files.

Example 83 includes the subject matter of any one of Examples 64-82, wherein the medical signal handling protocol includes Digital Imaging and Communications in Medicine (DICOM).

Example 84 includes the subject matter of any one of Examples 64-83, wherein the medical data file is an imaging medical data file.

Example 86 includes one or more computer-readable media comprising instructions stored thereon that, when executed, cause one or more processors to perform the method of any one of Examples 64-84.

Example 87 includes a product comprising one or more tangible computer-readable non-transitory storage media comprising computer-executable instructions operable to, when executed by at least one computer processor, enable the at least one processor to perform the method of any one of Examples 64-84.

Example 88 includes an apparatus including means to perform the method of any one of Examples 64-84.

What is claimed is:

1. An apparatus including a memory storing logic corresponding to a medical data forwarding engine (MDFE), and one or more processors to execute the logic to perform operations including:
accessing encoded medical information compliant with a medical signal handling protocol, the encoded medical information from a medical computing system of a medical facility network, wherein the encoded medical information includes one of a medical data file or a medical file fragment;
preprocessing the encoded medical information by making the encoded medical information compliant with a networking communication protocol compatible with a web interface of a network external to the medical facility network (external network); and
forwarding, after preprocessing, the encoded medical information to the external network based on the networking communication protocol.

2. The apparatus of claim 1, wherein the operations include determining the networking communication protocol prior to preprocessing.

3. The apparatus of claim 2, wherein the networking communication protocol includes at least one of WebSocket or Hypertext Transport Protocol Secure (HTTPS).

4. The apparatus of claim 1, wherein the networking communication protocol allows full-duplex communication using a Transmission Control Protocol (TCP) connection.

5. The apparatus of claim 1, wherein the networking communication protocol uses a Secure Socket Layer (SSL) encryption or a Transport Layer Security (TLS) encryption.

6. The apparatus of claim 1, wherein the networking communication protocol uses a Secure Socket Layer (SSL) handshake or a Transport Layer Security (TLS) handshake.

7. The apparatus of claim 1, wherein preprocessing further includes at least one of: compressing, decompressing, appending routing information, encrypting, decrypting, format conversion, or anonymizing.

8. The apparatus of claim 6, wherein preprocessing that comprises format conversion includes changing a file format of the encoded medical information from a first file format to a second file format different from the first file format to make the encoded medical information compatible with the networking communication protocol.

9. The apparatus of claim 8, wherein the first file format or the second file format includes one of: a Joint Photographic Experts Group (JPEG) format, a Portable Network Graphics (PNG) format, a Tagged Image File Format (TIFF) format, an MPEG format, a MPEG4 part 14 (MP4) format, an Audio Video Interleave (AVI) format, a QuickTime File Format (MOV) format, a Windows Media Video (WMV) format, or Flash Video (FLV) format.

10. The apparatus of claim 7, wherein anonymizing includes at least one of: removing personal identifying information (PII), replacing PII with a unique identifier, reducing information in the PII, or adding noise to the encoded medical information.

11. The apparatus of claim 1, wherein the operations further include determining, prior to preprocessing and based on at least one of metadata of the encoded medical information or pixel data of the encoded medical information, one or more parts of the encoded medical information to be preprocessed.

12. The apparatus of claim 1, wherein the operations further include determining, prior to preprocessing and based on a preconfiguration of pixel data corresponding to one or more parts of the encoded medical information to be changed, the one or more parts of the encoded medical information.

13. The apparatus of claim 1, wherein the one or more parts correspond to at least one of metadata of the encoded medical information or payload data of the encoded medical information.

14. The apparatus of claim 11, wherein the encoded medical information is first encoded medical information, and the one or more parts are one or more first parts, the operations further including, after determining the one or more first parts, determining one or more second parts of a second encoded medical information based on a determination of the one or more first parts, preprocessing the one or more second parts to make the encoded medical information compliant with the networking communication protocol, and forwarding, after preprocessing the one or more second parts, the second encoded medical information to the external network based on the networking communication protocol.

15. A routing system including:
a web interface circuitry;
a memory storing logic corresponding a medical data forwarding engine (MDFE); and
one or more processors coupled to the web interface circuitry to execute the logic to perform operations including:
accessing encoded medical information compliant with a medical signal handling protocol, the encoded medical information from a medical computing system of a medical facility network, wherein the encoded medical information includes one of a medical data file or a medical file fragment;
preprocessing the encoded medical information by making the encoded medical information compliant with a networking communication protocol compatible with a web interface of a network external to the medical facility network (external network); and
forwarding, after preprocessing, and through the web interface circuitry, the encoded medical information to the external network based on the networking communication protocol.

16. The routing system of claim 15, wherein the operations further include receiving a medical data file from the medical facility network, and fragmenting the medical data file to obtain medical file fragments therefrom, the encoded medical information corresponding to one of the medical file fragments.

17. A method to be performed at a processing circuitry of a medical facility network, the method including:
accessing encoded medical information compliant with a medical signal handling protocol, the encoded medical information from a medical computing system of the medical facility network, wherein the encoded medical information includes one of a medical data file or a medical file fragment;
preprocessing the encoded medical information by making the encoded medical information compliant with a networking communication protocol compatible with a web interface of a network external to the medical facility network (external network); and
forwarding, after preprocessing, the encoded medical information to the external network based on the networking communication protocol.

18. The method of claim 17, further including receiving a medical data file from the medical facility network, and fragmenting the medical data file to obtain medical file fragments therefrom, the encoded medical information corresponding to one of the medical file fragments.

19. The method of claim 18, wherein fragmenting includes fragmenting based on a size of the medical data file.

20. The method of claim 18, wherein the processing circuitry is a processing circuitry of a Picture Archiving and Communication System (PACS).

21. The method of claim 17, wherein the encoded medical information includes a first medical file fragment of a first medical data file, the operations further including routing the first medical file fragment and subsequent medical file fragments of the first medical data file on a fragment by fragment basis by routing the first medical file fragment to the external network before all fragments of the first medical data file are accessed by the one or more processor.

22. The method of claim 21, wherein the first medical data file is part of a plurality of medical data files from the medical facility network, and the first medical file fragment is part of a plurality of medical file fragments of at least some of the medical data files, the operations further including routing the medical file fragments of at least some of the plurality of medical data files, and remaining ones of the plurality of medical data files to the external network before receiving all file fragments of at least one of the plurality of medical data files.

23. The method of claim 16, wherein the medical signal handling protocol includes Digital Imaging and Communications in Medicine (DICOM).

24. One or more computer-readable media comprising instructions stored thereon that, when executed, cause one or more processors to perform the method of claim 16.

25. An apparatus including means to perform the method of claim 16.
